(19) United States
(12) Patent Application Publication
(10) Pub. No.: US 2003/0083640 A1
(43) Pub. Date: May 1, 2003

(54) IV Safety assembly

(76) Inventor: Rebekah R. Sadiow, Lincoln, RI (US)

Correspondence Address: Robert J. Doherty, 11 George St., Barrington, RI, 02806, US

(21) Appl. No.: 10/033,085

(22) Filed: Oct. 25, 2001

(51) Int. Cl. A61B 19/00

(52) U.S. Cl. 604/409

(57) ABSTRACT

A fluid delivery system including a fluid container and cannula assembly that in turn includes a separable tube portion formed of a material having a lower specific gravity than the fluid in the container that in turn becomes a float within the container to improve the visibility of the fluid level therein.

### FIELD OF THE INVENTION

This invention relates to the provision of a noticeable float including a colored and/or phosphorescent float in a fluid container such as an intravenous bag (IV bag) so that the solution level in such bag may be easily seen. Specifically, the invention is the provision of a connector or cannula assembly or portion thereof having a puncture tube with a separable portion which then becomes the float when this separable portion is broken off or otherwise removed from the connecting tube after that portion has been inserted into the bag through the seal provided therein. Generally, the connector is constructed of a colored plastic, which will ensure that the float is easily seen and will float on the fluid surface within the bag. Generally, the separable portion constitutes a sharpened end that may be solid or otherwise designed to prevent coring as the sharp end is pushed through the seal in the IV bag.

### BACKGROUND OF THE INVENTION

A recurring problem for caregivers of patients who are connected to an IV or other fluid dispensing devices is that it is sometimes difficult to determine the fluid level in the bag. Such bags are commonly formed from colorless flexible plastic material to form a fluid containing pouch which is then suspended by known means. A lower narrowed access area of the pouch is connected to a fluid dispensing assembly, i.e., a cannula assembly, such that the patient may be connected to intravenously to receive the fluid supply within the pouch. Since most fluids administered to patients are colorless and the bag itself is generally colorless, i.e., transparent except for descriptive indicia, it is extremely difficult to determine the fluid level present in the bag. It is, however, important for the caregiver to be able to quickly determine this level for a number of reasons including those that are safety related, convenience of replenishment and the like.

Accordingly, it would be advantageous to have a system in which a float is positioned within the bag interior and floats on the fluid surface therein and which float has a distinctive contrast appearance from the fluid contained therein, e.g., is colored and/or phosphorescent such that the caregiver can immediately determine the fluid level remaining in the bag by simply quickly looking at the float position within the bag.

The prior art patents which applicant is aware and which are of general background interest are: U.S. Pat. Nos. 2,850,211; 3,777,697; 3,942,526; 4,014,010; 4,639,251; 5,423,346; and 5,470,327. In addition, U.S. Pat. No. 5,5527,295 to Wing discloses a gravitational magnetic floating ball valve for use as a protective fluid cut off valve in an IV bag. The ball is, however, restrained within a compartment located adjacent the bottom of the IV bag or other compartment to provide fluids sealing. U.S. Pat. No. 4,675,020 to McPhee discloses a cannula assembly having a separable tube puncture portion which is housed but in an area in which there would be no need for a float. U.S. Pat. No. 4,589,879 to Pearson also discloses a cannula assembly in which a tube including a piercing tip that is separable from the tube after piercing the barrier seal of the container which system provides a positive indication that the barrier has, in fact, been pierced as well as the added advantage of a tamperproof indicator. Although Pearson mentions the use of plastic for a piercing tip, Pearson indicates that such be ideally constructed of stainless steel and further specifically fails to recognize the possibility of using a separable portion of a piercing tube as a float mechanism.

### SUMMARY OF THE INVENTION

The objectives of the present invention are accomplished by the provision of an easily monitor able fluid delivery system comprising a fluid container having fluid including an upper surface defining the liquid level therein, a cannula assembly and a fluid delivery means, said container having see-through walls and adapted for suspension for delivery of the fluid contained therein through an outlet having a liquid barrier seal, said cannula assembly having a hollow tube including an upper end adapted for communication with the fluid in said container and a lower end operatively associated with said fluid delivery means, said tube upper end having a separable portion of a lower specific gravity than that of said fluid and adapted to float at the liquid upper surface and be clearly visible through said container walls once said portion is passed through said seal and separated from said tube.

It should also be further brought out that although the present invention is described with specific reference to an IV bag or container, it is more generally applicable to containers carrying any flowable material including non-medical applications so long as a float which is easily discernible for those in attendance is provided and that such float originated as a portion of the assembly which connects the container to the dispensing system.

Other objects, features and advantages of the invention shall become apparent as the description thereof proceeds when considered in connection with the accompanying illustrative drawings.

### DESCRIPTION OF THE DRAWINGS

In the drawings which illustrate the best mode presently contemplated for carrying out the present invention:

FIG. 1 is a perspective view showing a somewhat simplified and stylized cannula assembly modified in accordance with the provisions of the present invention;

FIG. 1A is a perspective view of the medical IV bag to which the cannula assembly of FIG. 1 has been connected and the separable portion of the piercing tube thereof removed and allowed to float to the top surface of the fluid to provide a liquid level indicator therefor;

FIG. 2 is a perspective view showing an intermediate step in the insertion sequence;

FIG. 3 is a front elevational view similar to FIG. 2;

FIG. 3A is an enlarged partial sectional view of a portion of FIG. 3;

FIG. 4 is a front elevational view similar to FIG. 3 but showing the cannula assembly connected to the IV bag;

FIG. 5 is an enlarged view similar to FIG. 4 but showing how the cannula tube has penetrated the IV throat sealant barrier;

FIG. 6 is a view similar to FIG. 5 but showing the manipulative step of pressing the sides of the IV bag together to grasp the removable portion of the tube to separate such from the remaining portion thereof;

FIG. 7 is a front elevational view showing the float position on the top surface of the IV fluid;

FIG. 8 is a modified form of the invention in which the piercing tip of the cannula tube is removably positioned over the main portion thereof;

FIG. 8A is a sectional view of a modified form of FIG. 8;

FIG. 8B is a further modified form of the invention wherein the piercing tip is a removable solid point positioned over the main portion of the cannula tube;

FIG. 9 is an enlarged partial view of an IV through which the piercing tube of the cannula shown in the FIG. 8 embodiment has been inserted through the IV container barrier seal;

FIG. 10 is a view similar to FIG. 9 showing the manipulative step of pressing the sides of the IV bag together to remove the separable portion of the piercing tube from the remainder thereof. It being noted that FIG. 10 is rotated 90 degrees from the position shown in FIG. 9 assuming that the IV bag is of flattened configuration;

FIG. 11 is a view of a still further modification of the present invention in which the upper frangible portion of the cannula tube provides a solid pointed end and wherein the base of the tube is reinforced by molded stability enhancing wings in a configuration presently commercially available;

FIG. 12 is a front elevational view of an IV bag showing the cannula embodiment shown in FIG. 11 being inserted through the barrier seal portion thereof;

FIG. 13 is an enlarged view of a portion of FIG. 12 showing the cannula of the FIG. 11 embodiment disposed through the barrier seal;

FIG. 14 is a view similar to FIG. 13 but rotated 90 degrees and shows the manner in which the sides of the bag may be manipulated to grasp the frangible separable portion of the cannula tube to remove such so as to enable it to float to the surface of the IV fluid level contained in the bag;

FIG. 15 is a perspective view showing a still further modified form of the cannula construction of the present invention in which the removable portion is inserted within the connecting tube portion thereof and presents a solid piercing point;

FIG. 15A is a partial cross-sectional view of a further modification from that shown in FIG. 15; and

FIGS. 16A through 16I are stylized versions of various forms which the removable portion of the cannula tube may take in accordance with the present invention.

### DETAILED DESCRIPTION OF THE PREFERRED EMBODIMENTS

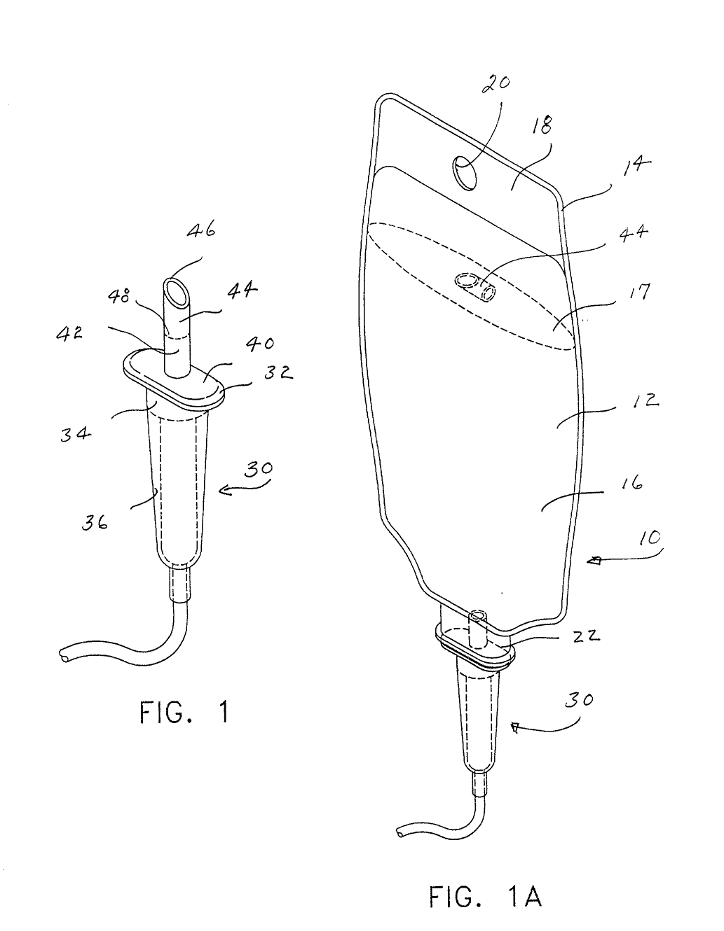

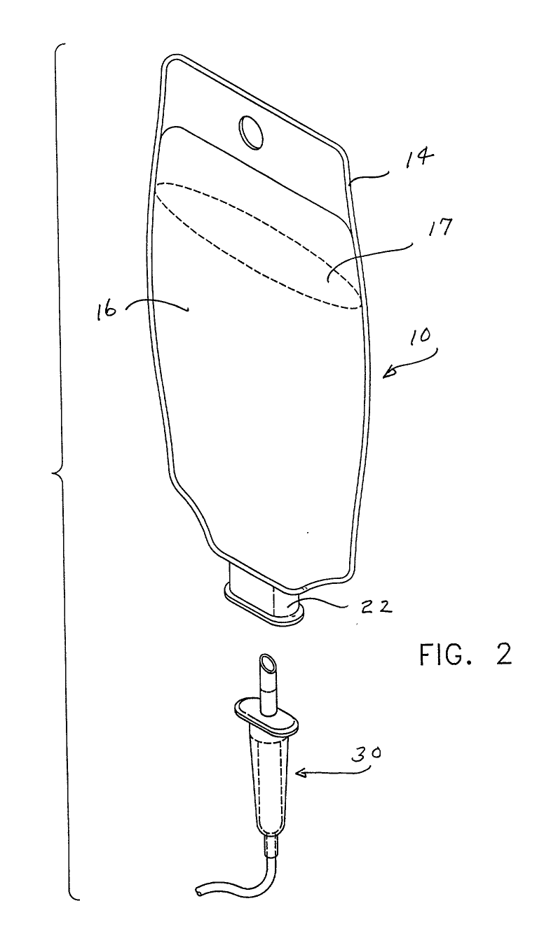

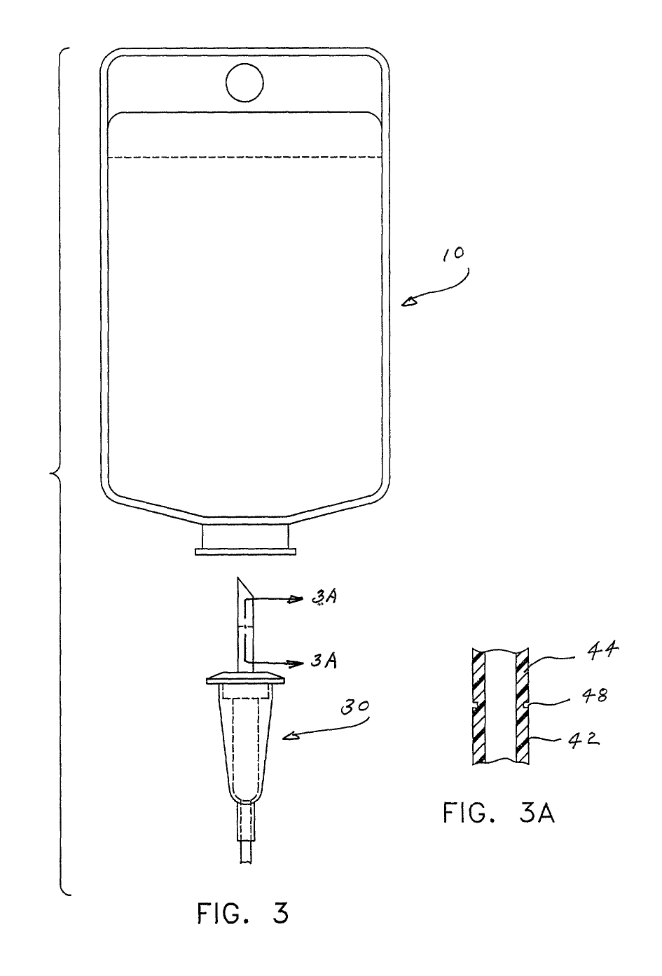

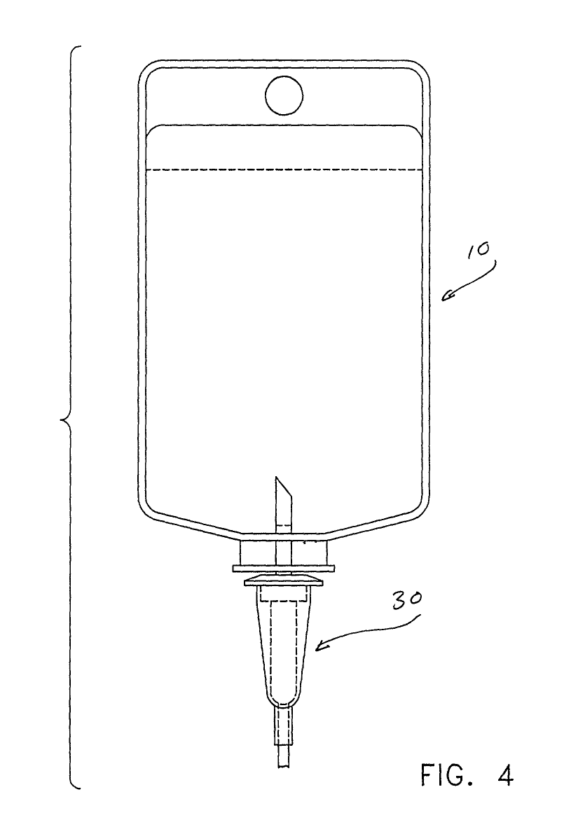

Referring now to the drawings and particularly FIGS. 1 through 7 thereof, the preferred form of the invention is depicted. Medical IV bag 10 of known construction is provided. Such bag includes a pair of opposed plastic sheets or panels 12 which are sealed around their periphery 14 to form an enclosed pouch to receive a fluid 16. At the top of the bag, the panels are superposed to form a flap 18 having an opening 20 therein such that the bag may be suspended in an inverted position from a hook or other support device adjacent the patient's bed as is known in the medical arts.

The lower portion of the bag 10 is provided with a narrow throat portion 22 having a barrier seal 24 provided at the opening 26 thereof. The barrier 24 may be formed from numerous materials such as cork, resinous plastic including the walls of the bag depending upon the fluids contained within the IV bag and the particular construction of the bag. It should be brought out that the particular construction of the IV bag as above indicated is not a critical feature of the present invention so long as it enables the features of the present invention to be carried out as will hereinafter be more fully apparent and including, for example, a see-through effect such that the upper surface or level 17 of the liquid 16 may be readily observed by medical help in attendance; and for the purposes of at least the FIGS. 1 through 7 configuration, that the side walls or panels be flexible to the extent that they can be manipulated so as to remove and separate that portion of the cannula assembly that will form the float mechanism of the present invention.

The cannula assembly 30 includes a body 32 having a lower portion 34 which is connected to a fluid reservoir 36 which in turn provides a lower connection with tubing 38 which form a part of a delivery system such that liquid contained within the IV container can flow through the cannula assembly from the IV bag and thence into the patient. The body includes a platform 38 including an upper surface 40 from which a hollow tube 42 upwardly extends. The tube 42 includes an upper portion 44 having a sharpened end 46. The purpose of the platform and body is to enable the cannula to be manipulated such that the sharpened end 46 of the tube 42 may be used to penetrate the barrier 24 of the IV bag 10 and to provide a seal.

In the embodiment of the invention shown in FIGS. 1 through 7, the cannula tube 42 includes an upper portion 46 that is separable therefrom along a weakened line 48. The cannula tube is formed from a suitable engineered plastic that is rigid enough such that a sharpened surface 44 may be provided thereon either simultaneously with the molding thereof or sharpened thereafter by a machine operation. In addition, the tube 42 may be formed simultaneously with the body 32 from the same engineered plastic. It is important and a feature of the present invention that such engineered plastic be lighter, that is, have a lower specific gravity than that of the IV fluid contained within the bag 10, such that when the separable portion 44 is removed once the sharpened end 46 thereof has been utilized to penetrate the barrier 24 and is removed from the remaining portions of the tube 42 that this separable portion 44 will assume a floating position on the upper surface of the liquid level 17 as shown in FIG. 1A. The separable portion 44 thus becomes the float.

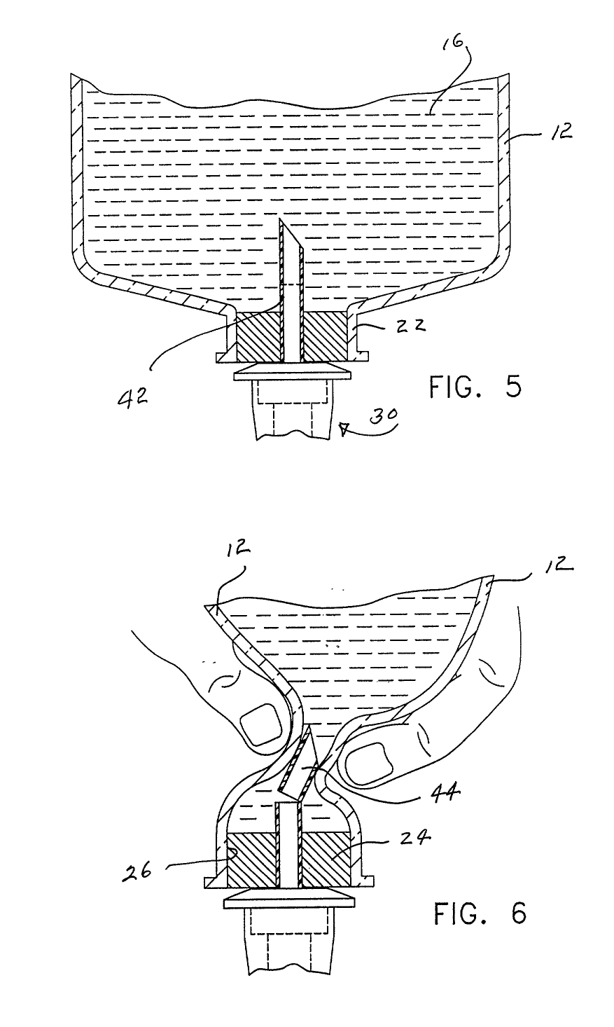

As shown in the transition between FIGS. 5, 6 and 7, it will be apparent that once the tube 42 penetrates the seal 24 and those portions of the cannula body provided for sealing contact with remaining portions of the barrier assure a sealing relationship between the cannula assembly and the IV bag (in the FIGS. 1 through 8 depiction, such may be provided by a portion of the top surface 40 of the shield), the caregiver may then inwardly manipulate the side panels 12 of the bag so as to firmly grasp the end 46 of the tube 44 and break such away from the remaining portion of the tube 42. It should be pointed out that the weakened line 48 to easily permit such could take a variety of forms including a peripheral score, a series of openings and the like so long as the separation of the portions 44 and 42 is facilitated by the above described action and that sufficient strength is present in the tube to enable the penetration previously described with respect to the barrier 24.

Generally the body of the cannula assembly is formed of a resinous plastic formed by injection molding procedures and is colored to contrast from the normally clear or at least transparent fluid delivery portion attached thereto and is further provided with a clear safety cap 50 to protect the user from the sharpened surface 46 as well as keeping such sanitary. This cap 50 can be easily removed prior to utilization of the assembly as above described. Thus, the normal color of the molded plastic forming the tube can serve as a contrast feature such that it is more easily observed by the caregiver floating at the upper level of the liquid 17. In addition, the tube 42 or at least the separable portion 44 thereof could additionally be provided with phosphorescent compounds molded therein during the molding procedure or applied thereafter that would further provide visual contrast for the float. It should also be apparent that changes necessary to conventional cannula assemblies in order to accomplish the goals of the present invention are minimal in that a presently commercially available cannula assembly can simply be provided with a weakened line 48 as above indicated.

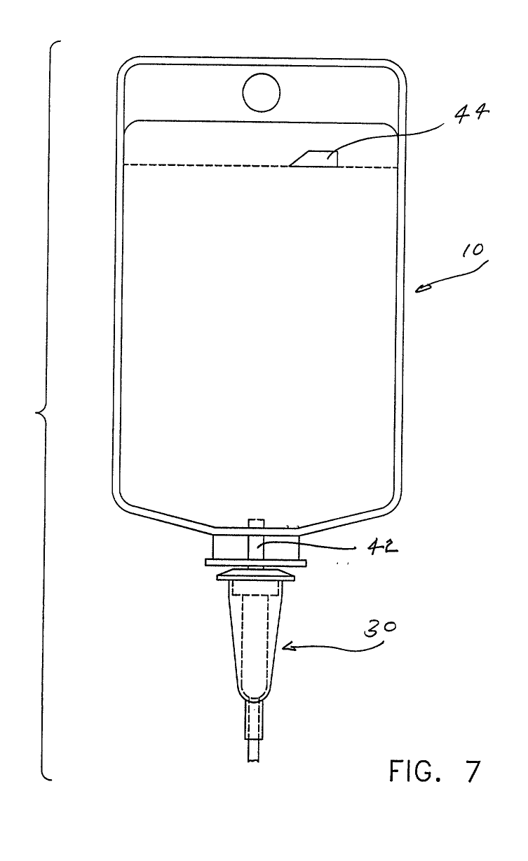

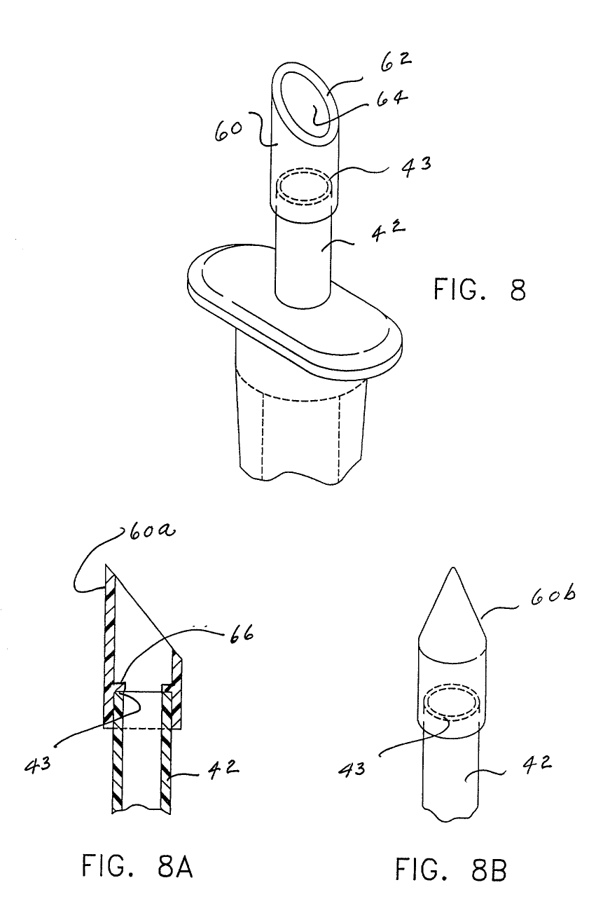

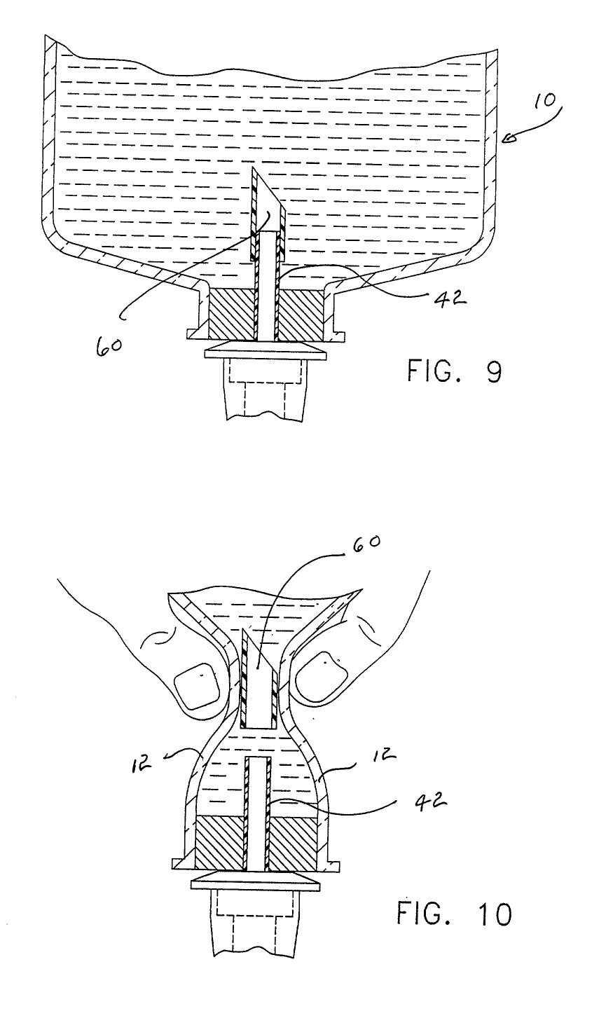

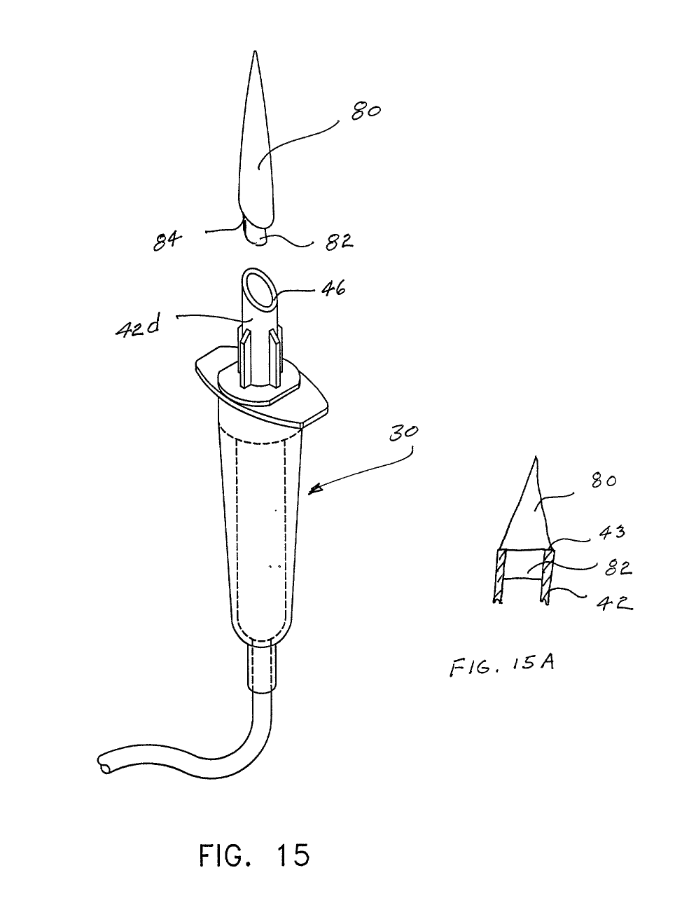

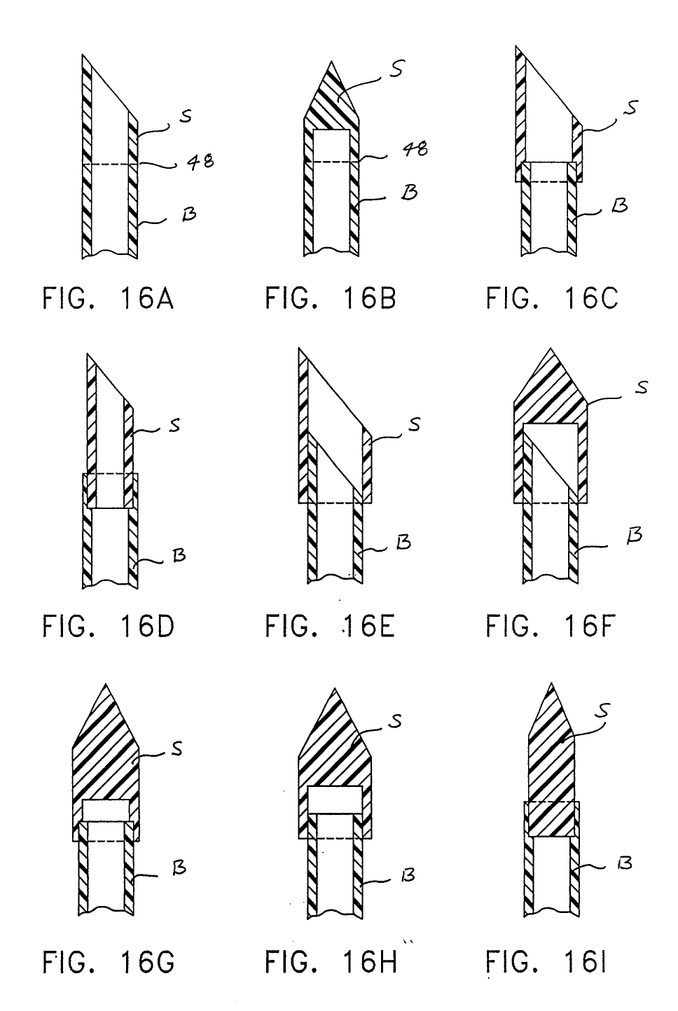

Turning now to FIGS. 8 through 10, a further embodiment of the present invention is described wherein the cannula tube 42 having upper surface 43 is provided with a removable cap 60 which terminates in a open sharpened upper edge surface 62 and includes an inner tubular surface 64 that is adapted to fit over the outside surface of the tube 42 and provide a friction fit thereon. In addition as will hereinafter be also apparent, instead of providing only a frictional surface to surface fit between the inside surface 64 of the cap 60 and the outside surface of the tube 42, an internal ledge 66 may be provided so as to contact the upper surface 43 of the tube 42 such that the downward force placed on the cap 60a when such is used to penetrate the barrier 24 will not tend to cause too great of a frictional interfit between the internal and external surfaces which could make it difficult to separate these two parts. In addition as shown in FIG. 8B, a cap 60b may be provided with a closed conical point 70 used to penetrate the barrier 24. Such a construction is useful to prevent coring from occurring when the tube is pushed through the barrier. This effect is undesirable as discussed in U.S. Pat. No. 4,589,879. In any of the above described cases and furthermore those that will be apparent from FIG. 16 hereinafter, the removable portion of the cannula tube 42 can be grasped by the caregiver by manipulating the sides 12 of the bag as shown in FIGS. 9 and 10 and thus free the separable portion that becomes the float so that the float may provide its visually noticeable position on the fluid surface 17.

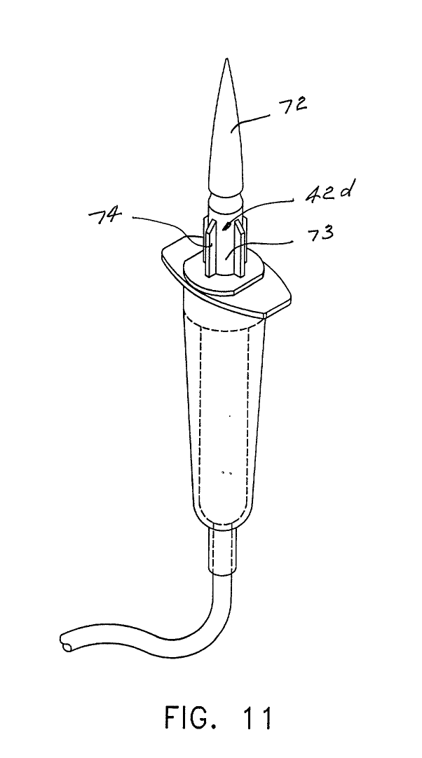

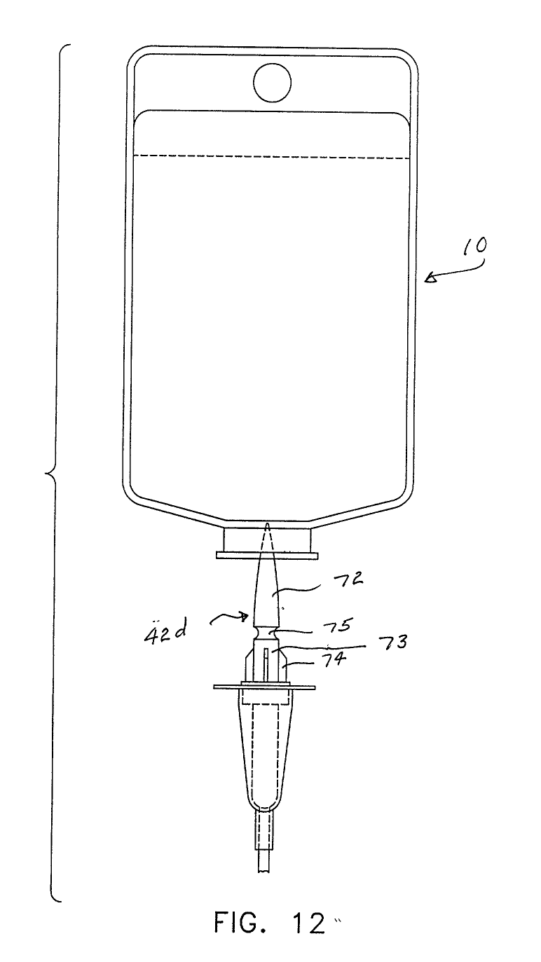

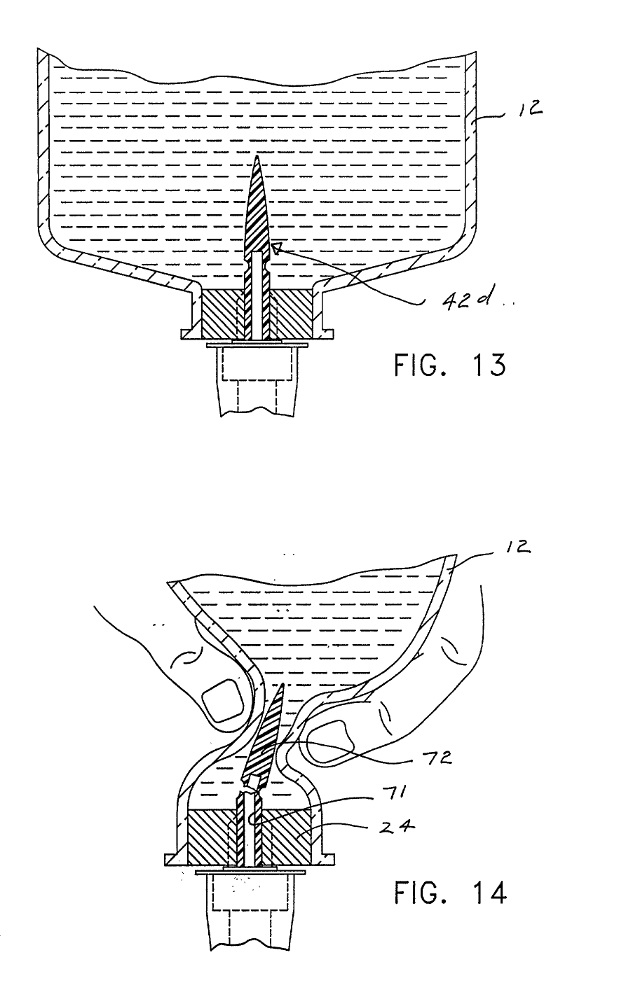

Turning now to FIGS. 11 through 14, a further embodiment of the present invention is shown wherein the cannula tube 42d is configured of an overall conical configuration having an internal duct or opening 71 that extends upwardly but terminates before a sharpened conically-shaped end 72 thereof. The tube 42d includes a base 73 including stiffening wings 74. A fracture joint 75 of a reduced circumferential extent or a weakened line is provided intermediate the tube but positioned beneath the upper extent of the channel or duct 71 such that once the point has been utilized to penetrate the barrier 24 and the effective sealing of that particular style cannula assembly is achieved vis-à-vis the IV bag, then the walls of the IV bag may similarly, as previously disclosed, be manipulated to grasp the upper portion of the of the tube (the conical point) and break such away from the lower portion 42d thereof and enable the removed conical point 72 to float to the surface 17. Obviously, the removable end 72 could be of configurations other than conical.

FIG. 15 describes a similar but different embodiment from that shown in FIGS. 11 through 14 in that a separable sharpened, i.e., conically-shaped, portion 80 is provided with a lower shank 82 that extends into the open end of either a cannula tube 42 having a slanted surface 46 which may be sharpened or one having a flattened upper surface 43 such that the shank 82 extends thereinto (see FIG. 15A). A shoulder 84 of the tubular portion 80 outwardly extends therefrom and serves to contact the upper surface of the cannula tube 42 (either surface 46 or 43) thus blocking the top opening thereof and enabling the assembly to penetrate the barrier 24 as previously explained and then be manipulated to enable the separable portion 80 to become the float. An advantage of the FIG. 15 type configuration is that a separable portion 80 can be supplied as an add-on feature to an already commercially available cannula assembly. In this regard, it should also be pointed out that the sharpened surface 46 shown in the various views is somewhat simplified and that compound edges, etc. may be required to achieve a sufficiently sharp piercing point and that any shoulder 84 adapted to rest thereon would need to be similarly shaped so as to firmly rest thereon.

FIGS. 16A through 16I show a variety of forms which the separable portion of the cannula tube S may take when viewed in place vis-à-vis the cannula tube base B and in cross-sectioned stylized form. FIG. 16A, in essence, depicts the form shown in FIGS. 1 through 7 while that shown in FIG. 16B is a variation thereof, and both FIGS. 16A and 16B include a weakened line 48 to enable breakage of the separable portion S from the base B. FIG. 16C is similar to the form shown in FIG. 8A but including an outset step rather than an inward flange while FIG. 16G shows a similar seating but with a conical top configuration. FIGS. 16D and 16I are similar in that a lower shank or post extends from the separable portion and extends into the base portion to seat on a shelf. FIGS. 16E and 16F show frictional connection between the separable and base portions but illustrating piercing tops of different styles while the FIG. 16H configuration is similar to that shown in FIG. 16F but wherein the base has a flat top surface.

While there is shown and described herein certain specific structure embodying this invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed is:

1. An easily monitor able fluid delivery system comprising a fluid container having fluid including an upper surface defining the liquid level therein, a cannula assembly and a fluid delivery means, said container having see-through walls and adapted for suspension for delivery of the fluid contained therein through an outlet having a liquid barrier seal, said cannula assembly having a hollow tube including an upper end adapted for communication with the fluid in said container and a lower end operatively associated with said fluid delivery means, said tube upper end having a separable portion of a lower specific gravity than that of said fluid and adapted to float at the liquid upper surface and be clearly visible through said container walls once said portion is passed through said seal and separated from said tube.

2. The fluid delivery system of claim 1, wherein said tube upper end separable portion is connected to said tube by a weakened line.

3. The fluid delivery system of claim 2, wherein said weakened line being a circumferential score at the juncture of said tube upper end and said separable portion.

4. The fluid delivery system of claim 1, wherein said separable tube portion has a sharpened upper end for piercing said fluid barrier.

5. The fluid delivery system of claim 4, wherein said separable tube portion is hollow and terminates in an open sharpened upper surface.

6. The fluid delivery system of claim 4, wherein said separable tube portion is partially hollow and terminates in a closed upper surface.

7. The fluid delivery system of claim 1, wherein said separable tube portion is made of a plastic material.

8. The fluid delivery system of claim 4, wherein said tube and said separable tube portion are simultaneously injection molded of the same plastic material.

9. The fluid delivery system of claim 1, wherein the fluid container is in the form of a pouch having opposed planar side panels formed of flexible plastic sheet material.

10. The fluid delivery system of claim 9, wherein the container is an IV bag.

11. A cannula assembly adapted for use with an IV bag containing fluid and having flexible see-through side panels and a lower fluid outlet having a barrier seal, said cannula assembly having a hollow tube including an upper end adapted for communication with the fluid in the bag and a separable portion connected to said tube upper end, said separable portion having a sharp upper end which is adapted to pass through said barrier seal and thereafter separated from said tube upper end so as to place said tube upper end in communication with the fluid in the bag, said separable portion formed of a material having a lower specific gravity than the fluid within the bag such that the separable portion forms a float which floats on the upper surface of the fluid within the bag.

12. The cannula assembly of claim 11, wherein said separable portion is formed of plastic.

13. The cannula assembly of claim 12, wherein the separable portion is colored.

14. The method of placing a float upon the liquid level surface of a fluid contained within a container of the type including flexible see-through opposed side walls and having an outlet having a liquid barrier seal with a cannula assembly having a hollow tube including an upper end adapted for communication with the fluid contained within the container and wherein the tube upper end further includes a separable portion having a sharpened upper end and formed of a material with a lower specific gravity than that of the fluid within the container, the steps comprising: forcing the sharpened upper end through the barrier to a position where the upper end of the tube is above the barrier, grasping the container side walls so as to grasp the separable portion and then removing the separable portion from the tube and releasing the separable portion.

15. The method of claim 14, wherein the separable portion and tube are connected to each other along a weakened circumferential line and the separable portion is broken off the tube with the grasping action.
